(19) United States
(12) Patent Application Publication
(10) Pub. No.: US 2019/0001099 A1
(43) Pub. Date: Jan. 3, 2019

(54) VASCULAR CATHETER

(71) Applicant: AYRSHIRE AND ARRAN HEALTH BOARD, Ayr (GB)

(72) Inventor: Christopher Smart, Morden (GB)

(21) Appl. No.: 15/748,640

(22) PCT Filed: Jul. 28, 2016
(86) PCT No.: PCT/GB2016/052315

§ 371 Date: Jan. 29, 2018

(30) Foreign Application Priority Data
Jul. 30, 2015 (GB) 1513473.7

(51) Int. Cl. A61M 25/00; A61M 1/36

(52) U.S. Cl. CPC A61M 25/003; A61M 2025/0031; A61M 25/0052; A61M 1/3659

(57) ABSTRACT

There is provided a catheter assembly wherein at least one of the arterial lumen and the venous lumen are radially displaceable in the transverse plane relative to the longitudinal axis of the catheter body between an insertion position and a drawing position. The maximum radial displacement in the transverse plane, between the radially displaceable arterial and/or venous lumen and the longitudinal axis of the catheter body is greater in the drawing position than in the insertion position. There is also provided a method of inserting and removing the catheter assembly into or from a blood vessel, and a method of treating blood using the catheter assembly.

The present invention relates to a catheter, in particular a vascular catheter suitable for use in chronic haemodialysis, acute haemodialysis, haemofiltration, or extracorporeal CO₂ removal. There is also provided a method of inserting and retracting the catheter from a human or animal body and a method of treating blood using the catheter of the present invention, in particular in a method of haemodialysis (chronic or acute), haemofiltration or extracorporeal carbon dioxide removal.

### BACKGROUND TO THE INVENTION

Renal failure is characterized by an inability of the kidneys to detoxify the blood. As an entity, it may be sub-classified by speed of onset into chronic, and acute sub-variants. The natural history, prognosis, and treatment of these two entities are very different.

Chronic renal failure (CRF) is characterised by a slow predictable, and mostly irreversible loss of kidney function, developing over years to decades. In the Western world it is largely a consequence of diabetes, high blood pressure, or an intrinsic renal disease. Because the condition progresses slowly and predictably, preparations can be made to anticipate the eventual need for dialysis or transplant. An artero-venous fistula may be fashioned, or as a bridging measure, the patient may be fitted with a long-term dialysis catheter. Typically, these catheters have blunt ends, which demand a complex insertion technique, need x ray guidance, and are “tunnelled” under the skin to reduce the risk of infection. Long term dialysis catheters generally stay in place for months, or even years.

In contrast, acute renal failure (ARF) develops within the course of hours to days, often on the background of previously normal kidney function. It may develop as a consequence of serious illness, dehydration, trauma, burns, or drug toxicity. The majority of acute renal failure is reversible upon treatment of the underlying cause. If untreated however, ARF rapidly leads to life threatening disturbances in pH and electrolyte homeostasis. In such instances haemodialysis, or haemofiltration must be initiated immediately, and this is usually accomplished by way of a short-term ‘acute’ dialysis catheter inserted into a central vein.

Acute dialysis catheters are distinct from chronic dialysis catheters in several ways. They are pointed to facilitate simple insertion, and their short indwelling duration means they do not need to be ‘tunnelled’ under the skin to avoid infection. Insertion does not require continuous x-ray guidance, and is often carried out by an intensive care physician rather than a dedicated renal physician. As such, acute dialysis catheters are faster and easier to insert in an emergency, making them appropriate for the management of acute renal failure. This invention is applicable to both acute and chronic haemodialysis catheters

Extracorporeal carbon dioxide removal is an emerging modality of treatment for patients with certain classes of respiratory insufficiency. Treatment is similar to dialysis, in that blood is removed, treated, and returned to the body. Because of the similarity, this invention could also be adapted to perform extracorporeal carbon dioxide removal.

All dialysis catheters contain two lumens, namely an ‘arterial’ lumen configured to aspirate blood from the blood vessel for treatment and a ‘venous’ lumen configured to eject treated blood back into the blood vessel. Both lumens are generally placed within a large central vein.

If the blood flow in a dialysis catheter is unreliable, it interferes with the continuity of treatment. The blood flow rates required for dialysis and haemofiltration are relatively high (200 to 450 ml/minute). This flow generates a region of low pressure in the vein around the apertures of the aspirating lumen. This vacuum may under certain circumstances, entrain the internal wall of the vein onto the apertures, causing an abrupt reduction or cessation of flow to the dialysis/haemofiltration machine. The dialysis/haemofiltration machine detects the pressure change, and stops the flow of blood. This temporarily relieves the vacuum, allowing the machine to be restarted, but often the problem recurs repeatedly. Repeated interruptions of flow reduce the efficacy of the treatment, and lead to blood stagnation and subsequent clotting in the dialysis/haemofiltration membrane. Clotting necessitates changing the circuit and membrane, resulting in prolonged interruption to dialysis and increased expense. Repeated disposal of the stagnant blood may eventually lead to anaemia in the patient.

Techniques used to improve poor flow in a dialysis catheter include manipulating or rotating the catheter, increasing the pressure in the vein by administration of intravenous fluid, or reversing the direction of flow through the catheter. These interventions may be repeated, and consume the time and attention of medical staff. Reversing the flow of blood also results in admixing of treated and untreated blood resulting in inefficient blood treatment/dialysis. Dialysis catheters, in particular central venous catheters are associated with a relatively high risk of infection and clotting, which can lead to low blood flow to the blood vessel and scarring and narrowing thereof.

There remains a need for catheter assemblies, which allow for rapid aspiration rates, without the associated problem of vessel wall entrainment occluding the apertures of the aspirating lumen.

The catheter assembly of the present invention is configured to ensure that the vein wall cannot be entrained onto the arterial lumen apertures which would result in complete or partial occlusion of said apertures. This ensures consistent blood flow through the arterial lumen with the associated benefits detailed below.
A) A reduction in the amount of downtime where the patient isn't receiving renal replacement therapy (RRT).
B) A reduction in the intensiveness of the medical intervention and labour required due to nurses and doctors having to manipulate unreliable dialysis lines less frequently.
C) A reduction in the number of repeated line insertions due to failure of an existing line.
D) A reduction in the loss of blood due to stagnation, clotting and subsequent disposal, thereby reducing the number of blood transfusions required and the risk of anaemia.
E) A reduction in the number of filter membranes required due to a reduction in blood stagnation and subsequent clotting in the dialysis machine.

There is also an advantage in one ideation of the design, in that the apertures of the device may be selectively occluded when the device is not in use. This may prevent blood tracking up inside the device, and clotting or forming intra-lumens fibrin deposits.

### STATEMENT OF INVENTION

According to a first aspect of the present invention, there is provided a catheter assembly, said catheter assembly comprising a catheter body defining an arterial lumen and a venous lumen, the catheter body including a distal tip portion including or housing a terminal catheter portion, the arterial lumen including at least one aperture at or towards the distal end of the arterial lumen and in fluid communication with the arterial lumen, and the venous lumen including at least one aperture at or towards the distal end of the venous lumen and in fluid communication with the venous lumen,
wherein the arterial lumen is radially displaceable in the transverse plane, relative to the longitudinal axis of the catheter body. This movement generally occurs as a laterally ‘bowing’ curvature over a section of length of the catheter towards the tip. There is convergence with the central axis, suitably at both ends of this section, and generally maximal radial displacement at mid-section. It is moveable between an insertion position and a drawing position, wherein the maximum radial displacement in the transverse plane, between the arterial lumen and the longitudinal axis of the catheter body is greater in the drawing position than in the insertion position. One of the arterial and venous lumens may form a helix around the central longitudinal axis, in which case the helix angle will be greater in the drawing position than in the insertion position. Alternatively, one of the arterial and venous lumens may laterally bow or curve along the central longitudinal axis.

Generally, the angle between the arterial lumen and the longitudinal axis of the catheter body at or towards the distal tip portion is greater in the drawing position than in the insertion position.

In the drawing position, some or all of said apertures of the arterial lumen generally face radially inwards towards the longitudinal axis of the catheter body, or are radially disposed around the arterial lumen within 30° (moving clockwise or anti-clockwise) from the cross-sectional plane which intersects the longitudinal axis of the catheter body and the wall of the arterial lumen facing towards the longitudinal axis of the catheter body. Typically, some or all of said apertures of the arterial lumen face towards the longitudinal axis of the catheter body, or within 15° (moving clockwise or anti-clockwise) from the cross-sectional plane; suitably within 10⁰; more suitably to within 5° from the cross-sectional plane. Generally, the majority of said apertures of the arterial lumen face towards the longitudinal axis of the catheter body, or within 30° (moving clockwise or anti-clockwise) from the cross-sectional plane which intersects the longitudinal axis of the catheter body and the wall of the arterial lumen facing towards the longitudinal axis of the catheter body.

According to a further aspect of the present invention, there is provided a catheter assembly, said catheter assembly comprising a catheter body defining an arterial lumen and a venous lumen, the catheter body including a distal tip portion including or housing a terminal catheter portion, the arterial lumen including at least one aperture at or towards the distal end of the arterial lumen and in fluid communication with the arterial lumen, and the venous lumen including at least one aperture at or towards the distal end of the venous lumen and in fluid communication with the venous lumen,
wherein the, or the majority of the aperture(s) at or towards the distal end of the arterial lumen face towards the longitudinal axis of the catheter body, or within 30° (moving clockwise or anti-clockwise) from the cross-sectional plane which intersects the longitudinal axis of the catheter body and the wall of the arterial lumen facing towards the longitudinal axis of the catheter body.

According to a further aspect of the present invention, there is provided a catheter assembly, said catheter assembly comprising a catheter body defining an arterial lumen and a venous lumen, the catheter body including a distal tip portion including or housing a terminal catheter portion, the arterial lumen including at least one aperture at or towards the distal end of the arterial lumen and in fluid communication with the arterial lumen, and the venous lumen including at least one aperture at or towards the distal end of the venous lumen and in fluid communication with the venous lumen,
wherein at least one of the arterial and venous lumens is disposed in a helix around the longitudinal axis of the catheter body and wherein the helix angle towards the distal tip portion is greater in the drawing position than in the insertion position.

Where the catheter is a chronic haemodialysis catheter, the method of insertion generally includes creating a subcutaneous tunnel, traversed by the catheter, before its insertion into the vein.

There is also provided a method of inserting any one of the catheter assemblies disclosed herein comprising:
making an incision in a blood vessel,
inserting the catheter body into the blood vessel.

If the arterial and/or the venous lumen is movable between an insertion and a drawing position, the arterial and/or venous lumen is in the insertion position upon insertion and/or upon removal of the catheter body into the blood vessel. The arterial lumen is moved into the drawing position prior to and upon the drawing of blood from the blood vessel.

According to a further embodiment, there is provided a method of removing any one of the catheter assemblies disclosed herein from a blood vessel comprising:
preventing blood from being drawn through the at least one aperture at or towards the distal end of the arterial lumen,
preventing blood from being returned through the at least one aperture at or towards the distal end of the venous lumen, and
removing the catheter body from the blood vessel.

According to a further embodiment, there is provided a method for treating blood comprising:
making an incision in a blood vessel,
inserting at least part of the catheter body into the blood vessel (generally the distal end of the catheter body),
drawing blood through the at least one aperture at or towards the distal end of the arterial lumen,
treating the blood,
returning the treated blood to the blood vessel through the at least one aperture at or towards the distal end of the venous lumen.

According to one embodiment, the method for treating blood is used to treat or mitigate the symptoms of low or inadequate renal function and/or renal failure. The method for treating blood is generally used in the context of renal replacement therapy. The method may be used for acute or chronic haemodialysis, or haemofiltration. According to a further embodiment, the method for treating blood is used for extracorporeal carbon dioxide removal.

According to a further embodiment there is provided a kit of parts comprising any one of the catheter assemblies as described herein and instructions for use. Generally, the catheter assembly is provided with the arterial lumen in the insertion position.

Where appropriate, teaching relating to any aspect or embodiment may relate to any other embodiment.

### Definitions

The distal end of the catheter assembly, the arterial lumen and the venous lumen is used to refer to the end of the catheter tube/arterial lumen/venous lumen which, in use, is positioned within a patient's body, generally within the patient's blood vessel.

The proximal end of the catheter assembly, the arterial lumen and the venous lumen is used to refer to the end of the catheter tube/arterial lumen/venous lumen which, in use, extends out of the patient.

An arterial lumen is configured to aspirate blood from the blood vessel for treatment and a venous lumen is configured to eject treated blood back into the blood vessel. In use, the arterial lumen and the venous lumen are positioned in a blood vessel, generally a vein.

At or towards the distal portion generally indicates less than 40% of the length of the catheter body nearest to the distal tip portion, typically 20% or less; suitably 10% or less, more suitably 5% or less of the length of the catheter between the distal tip portion and the deployment mechanism.

Extracorporeal membrane carbon dioxide removal is generally used to treat inadequate respiratory function or respiratory failure for instance in critically ill patients. The aim is to remove excess carbon dioxide from the blood for patients who receive oxygen by mechanical ventilation. A suitable method of extracorporeal membrane carbon dioxide removal involves drawing blood from a patient's circulation and passing it through a synthetic membrane, where carbon dioxide is removed, before it is returned to the circulation.

By an “effective” amount or “therapeutically effective amount” is meant an amount of one or more active substances which, within the scope of sound medical judgment, is sufficient to provide a desired effect without adverse effects, or other problem or complication, commensurate with a reasonable benefit/risk ratio.

All numerical values provided incorporate 10% less than and 10% more than the numerical value provided.

### DETAILED DESCRIPTION

Catheter Assembly

According to a first aspect of the present invention, there is provided a catheter assembly, said catheter assembly comprising a catheter body defining an arterial lumen and a venous lumen, the catheter body including a distal tip portion including or housing a terminal catheter portion, the arterial lumen including at least one aperture at or towards the distal end of the arterial lumen and in fluid communication with the arterial lumen, and the venous lumen including at least one aperture at or towards the distal end of the venous lumen and in fluid communication with the venous lumen,
wherein at least one of the arterial lumen and the venous lumen are radially displaceable in the transverse plane relative to the longitudinal axis of the catheter body between an insertion position and a drawing position; wherein in the drawing position, at least one of the arterial lumen and the venous lumen laterally curve relative to the longitudinal axis of the catheter body; wherein the maximum radial displacement in the transverse plane, between the radially displaceable arterial and/or venous lumen and the longitudinal axis of the catheter body is greater in the drawing position than in the insertion position.

Suitably, the radially displaceable arterial and/or venous lumen form a helix around the longitudinal axis of the catheter body.

Generally, the arterial lumen is radially displaceable in the transverse plane relative to the longitudinal axis of the catheter body wherein the angle between the longitudinal axis of the catheter body and the wall of the arterial lumen facing towards the longitudinal axis of the catheter body at or towards the distal tip portion is greater in the drawing position than in the insertion position. The curvature of the arterial lumen is typically greater in the drawing position than in the insertion position, the maximal separation between the arterial lumen and the central longitudinal axis is thus increased accordingly in the drawing position compared to the insertion position. Generally, the average separation between the arterial lumen and the central longitudinal axis is also increased in the drawing position compared to the insertion position. According to one embodiment, the maximum lateral distance between the arterial lumen and the longitudinal axis of the catheter body towards the distal tip portion is greater in the drawing position than in the insertion position.

In the drawing position, the majority or all of said apertures of the arterial lumen generally face towards the longitudinal axis of the catheter body, or within 30° (moving clockwise or anti-clockwise) from the cross-sectional plane which intersects the longitudinal axis of the catheter body and the wall of the arterial lumen facing towards the longitudinal axis of the catheter body. Typically, within 20° from the cross-sectional plane; generally, within 15°; suitably within 10°; more suitably to within 5° from the cross-sectional plane.

If the aperture(s) of the arterial lumen contact the wall of the blood vessel as blood is being drawn from the blood vessel, the arterial aperture(s) may become entrained onto the blood vessel walls, causing partial or complete occlusion of the aperture(s). The catheter assembly of the present invention overcomes or mitigates the problems associated with the rim of the aperture(s) of the arterial lumen being occluded by contact with the blood vessel wall. Such occlusion reduces or prevents blood flow through the arterial lumen making blood transfer from the blood vessel inefficient. The entrainment of the blood vessel wall onto the aperture rim can also cause damage to the blood vessel wall itself. The configuration of the catheter assembly of the present invention, and the particular positioning of the arterial aperture(s) prevents or reduces the risk of the rim of the arterial aperture(s) contacting the walls of the blood vessel, and thus becoming blocked. The blood flow through the arterial apertures, and consequently through the arterial lumen is therefore maximised, resulting in an efficient, effective method of removing blood from the blood vessel for treatment. Patients who have acute, or acute on chronic renal failure require renal replacement therapy urgently, and any inefficiency caused through blocking of the arterial apertures may affect the patient's prognosis.

In use, some or all of the apertures at or towards the distal end of the arterial lumen generally face away from the nearest blood vessel wall, typically at least 90°, suitably at least 150° from the cross-sectional plane which intersects the wall of the arterial lumen facing towards the blood vessel and the nearest blood vessel wall.

Generally the, or the majority of the apertures at or towards the distal end of the venous lumen face within 70 to 180° (moving clockwise or anti-clockwise) from the cross-sectional plane which intersects the longitudinal axis of the catheter body and the wall of the venous lumen facing towards the longitudinal axis of the catheter body, typically 80 to 110°, suitably around 90° from the cross-sectional plane.

The positioning of the venous and arterial apertures as described herein helps to maintain the aspirated and ejected blood separately and reduces the risk of mixing.

Generally, the angle between the arterial lumen and the longitudinal axis of the catheter body towards the distal tip portion is at least 10° greater in the drawing position than in the insertion position, suitably 15 to 30°.

Generally, the arterial lumen is affixed at the longitudinal axis of that catheter body at or towards the distal tip portion, and suitably affixed at another location along the longitudinal axis of the catheter body. According to one embodiment, the maximum lateral distance between the arterial lumen and the longitudinal axis of the catheter body towards the distal tip portion is at least two times greater in the drawing position than in the insertion position; typically, five to ten times greater. Generally, the maximal lateral distance between the arterial lumen and the longitudinal axis of the catheter body is at least 1 cm greater in the drawing position than in the insertion position.

According to one embodiment, the maximum lateral distance between the arterial lumen and the longitudinal axis of the catheter body towards the distal tip portion is 0.2 to 1.5 cm in the drawing position, and 0.1 cm or less in the insertion position.

According to one embodiment the maximum lateral distance between the arterial lumen and the longitudinal axis of the catheter body towards the distal tip portion is around 0.4 cm in the drawing position, and less than 0.5 mm in the insertion position.

Generally, where the catheter assembly is configured for use in a human, the greatest lateral distance between the arterial lumen and the longitudinal axis of the catheter body towards the distal tip portion is 1.0 to 1.5 cm in the drawing position, and 0.1 cm or less in the insertion position.

In the insertion position, the arterial lumen, and/or the venous lumen may be positioned substantially coincident with the longitudinal axis of the catheter body.

According to one embodiment, in the insertion position, the arterial lumen is disposed at an angle of no more than 5° from the longitudinal axis of the catheter body at or towards the distal tip portion and in the drawing position the arterial lumen is disposed at an angle of at least 10° from the longitudinal axis of the catheter body at or towards the distal tip portion.

According to one embodiment, the arterial lumen is disposed around the longitudinal axis of the catheter body in the form of a helix. The arterial lumen generally rotates about and translates along the longitudinal axis of the catheter body.

Typically, the venous lumen is also movable relative to the longitudinal axis of the catheter body between an insertion position and a drawing position wherein the angle between the venous lumen and the longitudinal axis of the catheter body towards the distal tip portion is greater in the drawing position than in the insertion position. Generally, the greatest lateral distance between the venous lumen and the longitudinal axis of the catheter body towards the distal tip portion is greater in the drawing position than in the insertion position.

According to one embodiment, the angle between the venous lumen and the longitudinal axis of the catheter body at or towards the distal tip portion is at least 10 to 15° greater in the drawing position than in the insertion position. The venous lumen may be disposed at an angle of no more than 5° from the longitudinal axis of the catheter body at or towards the distal tip portion and in the drawing position the venous lumen is disposed at an angle of at least 10° from the longitudinal axis of the catheter body at or towards the distal tip portion.

Suitably, the venous lumen is disposed around the longitudinal axis of the catheter body in the form of a helix. The venous lumen generally rotates about and translates along the longitudinal axis of the catheter body.

Generally, the arterial lumen and the venous lumen form a double helix around the longitudinal axis of the catheter body.

According to a further aspect of the present invention, there is provided a catheter assembly, said catheter assembly comprising a catheter body defining an arterial lumen and a venous lumen, the catheter body including a distal tip portion including or housing a terminal catheter portion, the arterial lumen including at least one aperture at or towards the distal end of the arterial lumen and in fluid communication with the arterial lumen, and the venous lumen including at least one aperture at or towards the distal end of the venous lumen and in fluid communication with the venous lumen,
wherein at least one of the arterial and venous lumens is disposed in a helix around the longitudinal axis of the catheter body; wherein the lumen(s) in the form of a helix is/are movable relative to the longitudinal axis of the catheter body between an insertion position and a drawing position and wherein the curve of the helix (es) towards the distal tip portion is greater in the drawing position than in the insertion position.

According to one embodiment, the arterial lumen is disposed in a helix around the longitudinal axis of the catheter body, suitably both of the arterial and venous lumens are disposed in a helix around the longitudinal axis of the catheter body. According to one embodiment, the arterial lumen and optionally the venous lumen are in the form of a helix when in the drawing position and when in the insertion position.

The venous lumen generally rotates about the longitudinal axis of the catheter body, typically in the same direction as the arterial lumen, and translates along the longitudinal axis of the catheter body, typically to approximately the same distance as the arterial lumen.

According to one embodiment, the arterial lumen and the venous lumen both rotate about the longitudinal axis of the catheter body in a clockwise direction. Alternatively, the arterial lumen and the venous lumen both rotate about the longitudinal axis of the catheter body in an anti-clockwise direction.

In the drawing position, typically the helix angle of the arterial and optionally the venous lumen(s) is at least 15° from the longitudinal axis of the catheter body at or towards the distal tip portion, generally 20 to 30°.

According to one embodiment, the arterial and venous lumens are disposed at around the same angle from the longitudinal axis in the drawing position. According to one embodiment, the arterial and venous lumens are disposed at around the same angle from the longitudinal axis in the insertion position.

The dimensions of the catheter body will depend on its intended use, in particular with regard to the type of animal/human to be treated and the blood vessel of interest.

According to one embodiment, the maximum radius of the helix at its most divergent point is 0.2 to 1.5 cm; generally, 0.5 to 1.2 cm in the drawing position, and 0.3 cm or less, generally 0.2 cm or less in the insertion position. Alternatively, the maximum radius of the helix at its most divergent point may be less than 1.2 cm, typically 0.4 to 1 cm in the drawing position, and 0.25 cm or less, typically 0.5 mm or less in the insertion position.

Typically, in the insertion position the angle between the arterial lumen and the longitudinal axis of the catheter body is no more than 3°, generally no more than 1°.

In the insertion position, both the arterial and venous lumens may be positioned substantially coincident with the longitudinal axis of the catheter body. The walls of the arterial and venous lumens facing the longitudinal axis of the catheter body may contact each other when in the insertion position.

According to one embodiment, one of the arterial and venous lumens may be disposed in a helix around the longitudinal axis of the catheter body, and the other of the arterial and venous lumens may be disposed substantially along the longitudinal axis of the catheter body. According to one embodiment, the venous lumen may be disposed in a helix around the longitudinal axis of the catheter body, and the arterial lumen may be disposed substantially along the longitudinal axis of the catheter body. The catheter assembly of the present invention may include more than one venous lumen, and two venous lumens may be disposed in a double helix around the longitudinal axis of the catheter body.

Where the arterial lumen is disposed substantially along the longitudinal axis of the catheter body, its cross-section may be greater than in other embodiments. In some contexts, such a catheter may be more accepted for use in extracorporeal membrane carbon dioxide removal. Where the venous lumen is disposed in a helix around the longitudinal axis of the catheter body, generally the, or the majority of the apertures at or towards the distal end of the venous lumen face within 70 to 120° (moving clockwise or anticlockwise) from the cross-sectional plane which intersects the longitudinal axis of the catheter body and the face of the venous lumen facing towards the longitudinal axis of the catheter body, typically 80 to 110°, suitably around 90° from the cross-sectional plane. Such positioning of the venous aperture(s) imparts a spiraling flow of blood, which may have additional benefits in terms of stability in the vein, and “splinting” the vein open.

Generally, the helix formed by the lumen(s) extends more than 20% along the length of the catheter body intended to be positioned within the patient's body in use (generally the distance from the distal tip portion to the deployment mechanism), typically 25 to 30%. The helix formed by the lumen(s) may extend 10 to 100 mm of the longitudinal axis from the distal tip portion of the catheter body, typically 40 to 60 mm, suitably around 50 mm from the distal tip portion. Generally, the lumen(s) rotates more than 90° around the longitudinal axis of the catheter body, typically 120° to 200°, suitably around 180° around the longitudinal axis of the catheter body. Generally, the lumen(s) do not rotate more than 250° around the longitudinal axis of the catheter body.

According to one embodiment, the helix angle is 10 to 20°. Generally, the helix performs a full revolution about the longitudinal axis of the catheter body every 5 to 10 cm.

When in the drawing position, the venous and arterial lumens generally bow, or arch, laterally in radially opposite or substantially opposite directions from the centreline of the longitudinal axis of the catheter body, suitably by virtue of a slight shortening of the longitudinal distance the lumens extend. Generally, the lumens bow or arch in directions which differ by at least 90°, typically at least 120°, suitably 150 to 200°, more suitably around 180° from the centreline of the longitudinal axis of the catheter body.

According to one embodiment, at any one location in the cross-section of the helix, the lumens splint two opposed sides, generally two directly opposed sides. Accordingly, if the helix is over expanded slightly, the blood vessel will be stretched into an oval shape, and this reduces the risk of excessive wall tension.

The maximum lateral spacing between the arterial and venous axes (generally the maximum radius of the helix at its most divergent point) is typically more than 10% of the diameter of the blood vessel into which the catheter assembly is intended to be inserted when the blood vessel is in its collapsed state. Typically, the maximum lateral spacing is 10 to 150% of the diameter, generally 10% to 50%; suitably 25 to 50%; more suitably around 40% of the diameter of the blood vessel in its collapsed state.

According to one embodiment, the spacing between the arterial lumen in the drawing position and the venous lumen is generally great enough to hold the blood vessel open (although this is not necessary for its function). Accordingly, the catheter assembly of the present invention allows increased blood flow into the arterial lumen during a blood treatment method, and promotes high flow of treated blood to be ejected into the blood vessel through the venous lumen.

Central veins in a human will generally range from 1 to 2 cm in a collapsed state. Veins are elastic, so stretch physiologically to about 150% of their normal diameter when slightly pressurised. Accordingly, it is not unreasonable for the maximum lateral spacing between the arterial and venous lumens to be 1.0-1.4 cm in the drawing position.

According to one embodiment, the arterial and venous lumens are maximally laterally spaced at a first longitudinal distance from the distal tip portion, and the apertures in the arterial lumen are at a second longitudinal distance from the distal tip portion, the first longitudinal distance being greater than the second longitudinal distance.

Because blood in veins flows towards the heart, and the catheter body of the present invention is generally inserted into a vein peripherally, blood flow down the vein is away from the proximal end and towards the distal tip portion. Therefore, for maximum efficiency, it is advantageous to aspirate blood from higher upstream, and inject it further downstream. Otherwise the same blood is dialysed twice (recirculation). Therefore, the arterial (aspirating) apertures are generally further from the distal tip portion than the venous (ejecting) apertures.

The lateral distance between the arterial lumen and the longitudinal axis of the catheter body may be greater in the drawing position than in the insertion position for at least 25 to 60% of the length of the catheter body intended to be positioned within the patient's body in use, generally 30 to 40%. Generally, the lateral distance between the arterial lumen and the longitudinal axis of the catheter body may be greater in the drawing position than in the insertion position for at least 25 to 60% of the distance between the distal tip portion and the deployment mechanism.

The helix may extend longitudinally for at least 5 mm from the distal tip portion, generally at least 10 mm.

According to one embodiment, the catheter body defines a third lumen extending from the distal tip portion substantially along the longitudinal axis of the catheter body.

The third lumen suitably decreases the amount of lateral flexibility of the catheter assembly during insertion into a patient's body, and into a blood vessel. This may be due to the inflexibility of the third lumen itself, or due to the incorporation of a removable stiffening member within the third lumen. Accordingly, the incorporation of the third lumen can increase the ease of insertion of the catheter body. Generally, the distal end of the third lumen is housed within the distal tip portion, and the proximal end of the third lumen is configured to extend out of the patient's body in use.

According to one embodiment, the catheter assembly of the present invention includes a deployment mechanism configured to move the arterial lumen (and optionally the venous lumen) between the insertion and the drawing position. Typically, the proximal end of the third lumen extends past the deployment mechanism; suitably the proximal end of the third lumen extends to approximately the same distance as the proximal end of the arterial lumen. The third lumen is generally configured to receive a guide wire there through. The third lumen is generally inflexible enough to be configured to stretch the arterial and venous lumens along the longitudinal axis of the catheter body. This inflexibility of the third lumen may be a feature of the lumen itself, or the inflexibility of the third lumen may be increased by incorporation of a stiffening member within the third lumen. The catheter body generally remains flexible enough to flex, generally up to 20° over its length, typically up to 15° over its length.

Where a stiffening member is housed within the third lumen during insertion into a blood vessel, the stiffening member is generally (although not necessarily) removed from the third lumen following insertion prior to the drawing of blood.

According to one embodiment, the third lumen may be configured to allow administration of fluids (for instance medicaments) there through.

According to one embodiment, the walls of the arterial and venous lumens facing the longitudinal axis of the catheter body contact the third lumen when in the insertion position.

Suitably the arterial and venous lumens, and optionally the third lumen are fixedly housed within the distal tip portion. In particular the arterial and venous lumens, and optionally the third lumen generally maintain their positioning within the distal tip portion during deployment of the lumen(s) between the insertion and the drawing position.

One or more of the arterial, venous and/or third lumens generally have a circular or oval cross section.

According to one embodiment, the third lumen has a generally circular cross section, and the arterial and venous lumens have a C-shaped cross section, typically configured to house the third lumen within the inner walls of the lumens when the lumens are in the insertion position

The arterial and venous lumens typically include a plurality of apertures at or towards the distal end of the lumens in case one or more of the apertures is narrowed or blocked through clot formation. In addition, the incorporation of a plurality of lumens reduces the speed and turbulence of flow (therefore the pressure difference) through each aperture. Generally, each of the venous and arterial lumens include 2 to 10 apertures, typically 2 to 5 apertures.

The catheter assembly generally includes a deployment mechanism, configured to move the arterial lumen (and optionally the venous lumen) between the insertion and the drawing position. The skilled man would be aware of numerous suitable mechanisms including switch, twist and compression mechanisms.

The deployment mechanism generally moves the arterial lumen (and optionally the venous lumen) from the insertion to the drawing position by reducing the distance between the distal tip portion and the deployment mechanism. The decrease in distance is dependent on the angle and the lateral distance required between the arterial lumen and the longitudinal axis of the catheter body at or towards the distal tip portion, and in some embodiments the helix angle required. The greater the decrease in distance between the distal tip portion and the deployment mechanism, the greater the lateral distance between the arterial lumen and the longitudinal axis of the catheter body. Likewise, the greater the decrease in distance between the distal tip portion and the deployment mechanism, the greater the helix angle. The decrease in distance is generally 1 to 10 mm, typically around 5 mm.

According to one embodiment, the deployment mechanism moves the arterial lumen (and optionally the venous lumen) from the insertion to the drawing position through the application of a tractive force to the third lumen away from the distal tip portion. This generally causes the third lumen to move past or through the deployment mechanism.

According to one embodiment, the deployment mechanism is a twist mechanism, and a tractive force is applied to the third lumen by twisting the deployment mechanism, typically through 90°, to pull the third lumen a controlled distance (typically less than 10 mm, suitably around 5 mm).

According to a further embodiment, there is innate elasticity in the third lumen, but it is not free to move within the main body of the catheter. The catheter assembly may be configured such that it is in the drawing position. During insertion the catheter may be twisted such that the maximum radius of the helix at its most divergent point is reduced. When it is in position for deployment, rotating it in the other direction facilitates the maximum radius of the helix at its most divergent point increasing and the lumen(s) are moved into the drawing position

The catheter may include securement means adapted to secure the lumen(s) in the insertion position until the deployment mechanism is activated.

The proximal ends of the venous and arterial lumen are generally suitable for attachment to a dialysis machine, or to an extracorporeal carbon dioxide removal machine.

According to one embodiment, the catheter arrangement includes one radially displaceable arterial lumen, one radially displaceable venous lumen, and a central lumen disposed substantially along the longitudinal axis of the catheter body, where the central lumen does not take part in dialysis. Generally, the arterial lumen and the venous lumen are symmetrically or substantially symmetrically disposed along the longitudinal axis of the catheter body. The arterial and venous lumens are generally similarly dimensioned. The arterial and venous lumens may be in the form of a double helix around the central longitudinal axis of the catheter body. The central lumen in this embodiment suitably has a smaller cross-sectional area, and serves to deploy the opening mechanism, accommodate the wire during insertion, and may be used for fluid or drug administration.

According to another embodiment the catheter either aspirates or injects from both of the radially displaceable lumens, and blood flows in the contrary direction in the central lumen disposed along the longitudinal axis. The catheter includes two lumens radially displaceable in the transverse plane relative to the longitudinal axis of the catheter body between an insertion position and a drawing position. Generally, these two lumens are symmetrically or substantially symmetrically disposed along the longitudinal axis of the catheter body, generally arcing along the longitudinal axis when in the drawing position. The two lumens are generally similarly dimensioned, suitably having the same maximum radius or diameter of cross-section (or within 10%). The two lumens may be in the form of a double helix around the longitudinal axis of the catheter body. The two radially displaceable lumens may both be arterial lumen. Alternatively, the two radially displaceable lumens may both be venous. Advantageously, the lateral lumens are arterial and the central lumen is venous.

In this embodiment, a third lumen is mandated, and is disposed along the longitudinal axis of the catheter body. This may be the venous lumen, or the arterial lumen; advantageously the venous lumen. This lumen may be enlarged compared to the two lumens radially displaceable in the transverse plane relative to the longitudinal axis of the catheter body. The cross-sectional area of the lumen disposed along the longitudinal axis may be greater than the cross-sectional area of the lumens radially displaceable in the transverse plane relative to the longitudinal axis of the catheter body, suitably at least 20% greater, typically more than 50% greater. Suitably the central lumen is circular in cross section.

According to a further embodiment, the venous and arterial lumens are coaxial in both the insertion and drawing positions (one is typically housed or nested within the other). Suitably, the outer wall of the inside lumen (the lumen provided towards the longitudinal axis of the catheter), is in contact with the blood flow within the outside lumen. Such an arrangement saves space in cross section allowing a higher flow for a given amount of cross sectional area of the catheter. The amount of material required to form the catheter is also reduced. On pulling the inner lumen back, longitudinally, or helically oriented apertures in the form of slits in the distal section of the outer lumen are opened. This opening occurs by a lateral, or helically outward bowing motion of the split wall of the outer lumen over the distal segment. When the slits are opened, the outer lumen is in fluid communication with the blood stream, when the slits are closed, there is no communication. Generally there are two to six slits, suitably two. Alternatively, the wall of the outer lumen, at or towards the distal portion, may be thinned, or molded into several longitudinal ‘struts’ and when deployed, serve to form a kind of enlargeable basket, from which the outer lumen aspirates. In this embodiment, the outer lumen is in fluid communication with the blood stream in both the insertion and drawing position,

Method of Insertion and Removal

There is also provided a method of inserting any one of the catheter assemblies disclosed herein comprising:
making an incision in a blood vessel,
inserting the catheter body into the blood vessel wherein the arterial lumen is provided in the insertion position,
moving the arterial lumen into the drawing position.

The catheter assembly of the present invention may be suitable for use on a human or animal body. In particular, for use on a vein in the neck such as the internal jugular vein, chest such as the subclavian vein, or groin such as the femoral vein. The blood vessel is generally the internal jugular vein, a subclavian vein or a femoral vein.

According to one embodiment, a guide wire is inserted into the blood vessel prior to insertion of the catheter body in order to reduce the risk of incorrect positioning of the catheter body. Where the catheter assembly includes a third lumen, the third lumen is generally configured to house the guide wire during insertion of the catheter body. In such embodiments, the method includes inserting the catheter body into the blood vessel such that the guide wire is housed within the third lumen.

Alternatively, the venous lumen may be configured to receive the guide wire. This is particularly appropriate where the venous lumen extends substantially along the longitudinal axis of the catheter body. In such embodiments, the method includes inserting the catheter body into the blood vessel such that the guide wire is housed within the venous lumen.

The catheter body may be inserted into the blood vessel using the Seldinger technique. In general terms this involves inserting a needle into a vein, inserting a guide wire into the vein through the needle, optionally nicking the skin next to the guide wire (typically with a knife or equivalent), dilating a tract through soft tissues into the vein using a dilator wherein the guide wire is generally housed within the dilator, removing the dilator and sliding the catheter body of the present invention over the guide wire, generally through the central lumen.

Generally after insertion of the catheter into the blood vessel, the guide wire is removed.

The longitudinal axis of the catheter body is generally inserted into the blood vessel at an angle of 10 to 45° from the longitudinal axis of the blood vessel.

Where the catheter is a chronic haemodialysis catheter, the method of insertion may include the formation of a subcutaneous tunnel and the optional use of a removable (suitably tearable insertion sheath).

According to a further embodiment, there is provided a method of removing the catheter assembly disclosed herein from a blood vessel comprising:
preventing blood from being drawn through the aperture(s) at or towards the distal end of the arterial lumen,
preventing blood from being returned through the aperture(s) at or towards the distal end of the venous lumen,
moving the arterial lumen into the insertion position,
where the venous lumen is movable between an insertion position and a drawing position, moving the venous lumen into the insertion position; and
removing the catheter body from the blood vessel.

Method of Treating Blood

According to a further embodiment, there is provided a method for treating blood comprising:
making an incision in a blood vessel (typically using the Seldinger technique as detailed above),
inserting at least the distal end of the catheter body of the catheter assembly as disclosed herein into the blood vessel, wherein the arterial lumen is provided in the insertion position, and where the venous lumen is movable between an insertion position and a drawing position, the venous lumen is provided in the insertion position;
moving the arterial lumen and optionally the venous lumen into the drawing position, drawing blood through the at least one aperture at or towards the distal end of the arterial lumen,
treating the blood,
returning the treated blood to the blood vessel through the at least one aperture at or towards the distal end of the venous lumen.

Where the venous lumen is movable between an insertion position and a drawing position, the venous lumen should also be provided in the insertion position upon insertion.

The blood may be treated by the removal of waste products and/or extra fluid, typically which build up in the blood when the patient's kidneys are not functioning properly.

The method of treating blood may be used in renal replacement therapy, in particular in the treatment of inadequate renal function, including renal failure. The method may be used in the emergency treatment acute renal failure or acute on chronic renal failure.

According to a further embodiment, the blood may be treated to remove excess carbon dioxide. Typically, such extracorporeal carbon dioxide removal is effected by passing the blood through a membrane, suitably a synthetic membrane.

The method of treating blood may be used in the treatment of symptoms associated with reversible hypercarbic respiratory failure. Alternatively, or additionally, such extracorporeal carbon dioxide removal may be considered for patients being considered for lung transplantation. Patients for whom extracorporeal carbon dioxide removal would be of benefit have generally undergone prolonged mechanical ventilation.

The rate of blood flow drawn through the arterial lumen is generally 400-800 mL per minute.

The specific configuration of the catheter of the present invention reliably allows high rates of blood flow with a reduced risk of occlusion of the arterial lumen.

According to one embodiment, the catheter of the present teachings is used in the administration of medicaments or other substances to the blood vessel of the patient. Typically the medicaments and/or other substances are introduced via the third lumen of the catheter assembly.

The medicaments are generally administered in a therapeutically effective amount. Suitable medicaments include sterilized agents, antithrombin agents, anticoagulant agents, and antimicrobial agents.

Alternatively or additionally the third lumen may be used in the measurement of the central venous pressure.

The method of treating blood may include the method of insertion and/or the method of removal as described herein.

Kit of Parts

According to a further aspect of the present invention there is provided a kit of parts comprising the catheter assembly as described herein, and instructions for use. Generally, the catheter assembly is provided with the lumen(s) in the insertion position.

Generally, the catheter assembly is sterilized prior to being placed within the closed packaging and the closed packaging of the kit of parts maintains the sterility of its contents (including the catheter assembly) during storage. The catheter is generally used within 5 years of sterilization; typically, within 3 years from sterilization.

The kit of part may include one or more connection devices configured to attach the proximal end of the lumen(s) to a dialysis machine, or to a machine suitable for extracorporeal carbon dioxide removal.

Generally, the kit includes instructions for use, for example the nature of insertion, the method of moving the lumen(s) from the insertion to the drawing position, and details of a preferred method of insertion and removal.

Throughout the description and claims of this specification, the singular encompasses the plural unless the context otherwise requires. In particular, where the indefinite article is used, the specification is to be understood as contemplating plurality as well as singularity, unless the context requires otherwise. The words “comprise” and “contain” and variations of the words, for example “comprising” and “comprises”, mean “including but not limited to”, and are not intended to (and do not) exclude other moieties, additives, components, integers or steps. All documents referred to herein are incorporated by reference. Features, integers, characteristics, or groups described in conjunction with a particular aspect, embodiment or example of the invention are to be understood to be applicable to any other aspect, embodiment or example described herein unless incompatible therewith.

The present invention is further described by way of example only with reference to the accompanying Figures in which:

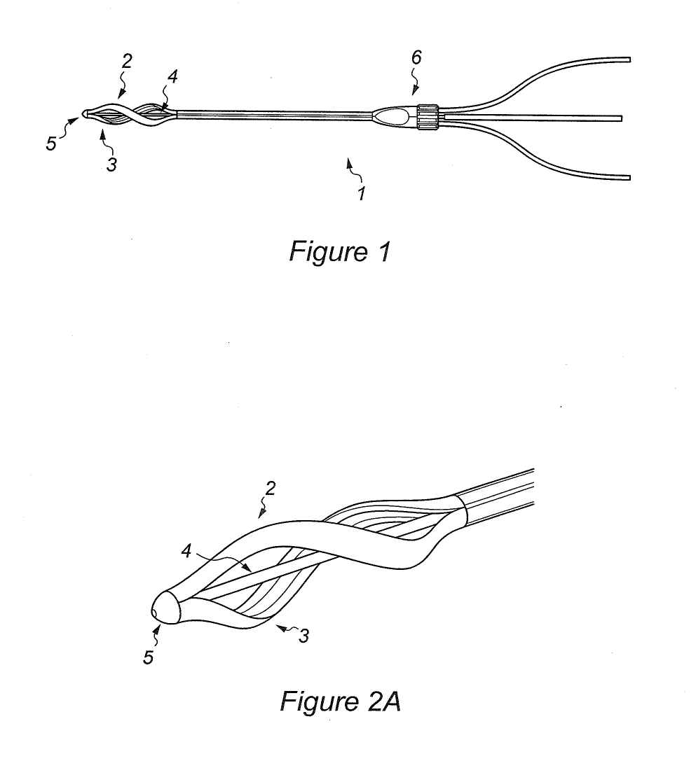

FIG. 1 provides a schematic representation of an embodiment of the catheter assembly of the present invention in the drawing position;

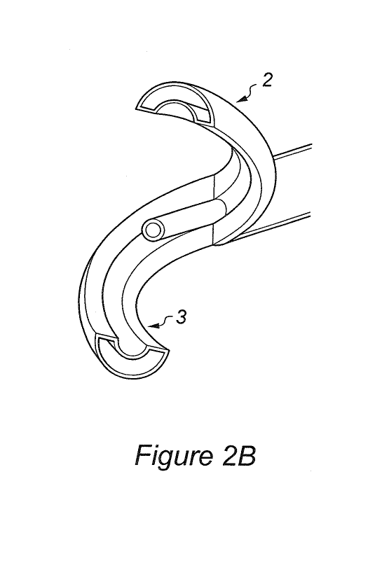

FIG. 2A provides a schematic representation of the dual helix formed by the arterial and venous lumens of an embodiment of a catheter assembly of the present invention;

FIG. 2B provides a cross section of the dual helix shown in FIG. 2A;

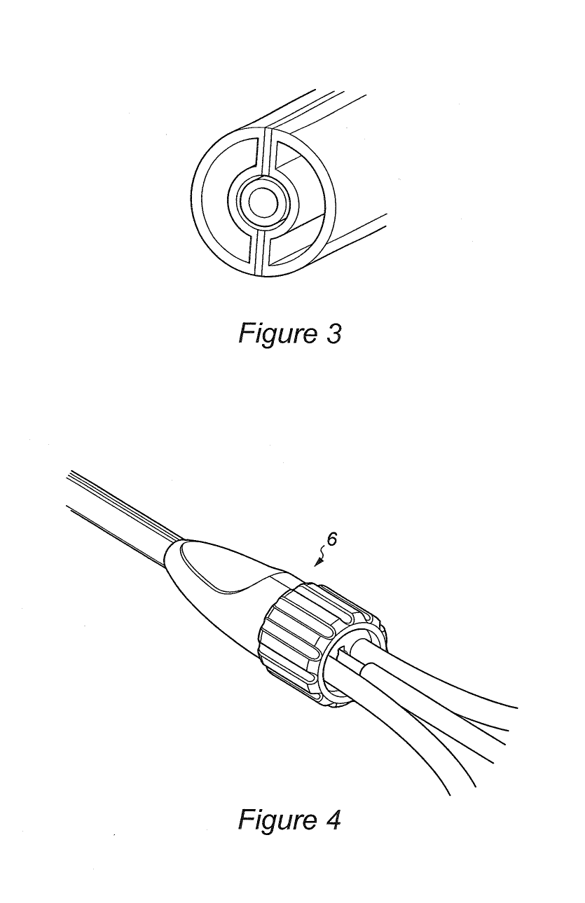

FIG. 3 provides a cross section of the lumens in the insertion position;

FIG. 4 provides a schematic representation of the rotating deployment mechanism with the central lumen pulled back through the deployment mechanism to illustrate how rotation of the threaded mechanism is translated into longitudinal distraction;

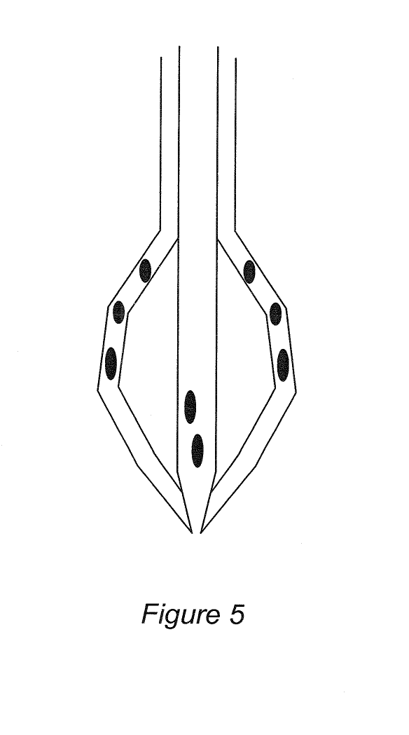

FIG. 5 provides a longitudinal cross-section of a catheter assembly of in the drawing position;

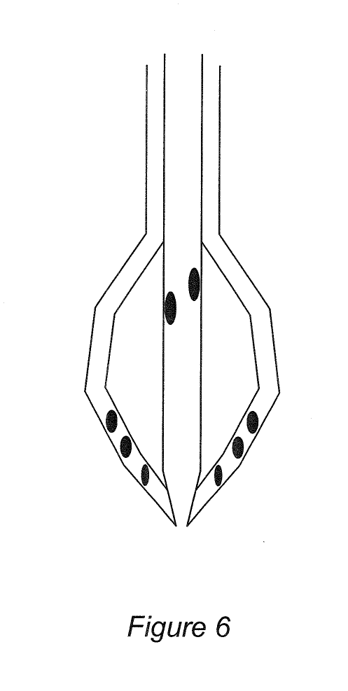

FIG. 6 provides a longitudinal cross-section of a catheter assembly of the present invention in which two venous lumens are disposed symmetrically about an arterial lumen, the illustrated catheter assembly is in the drawing position;

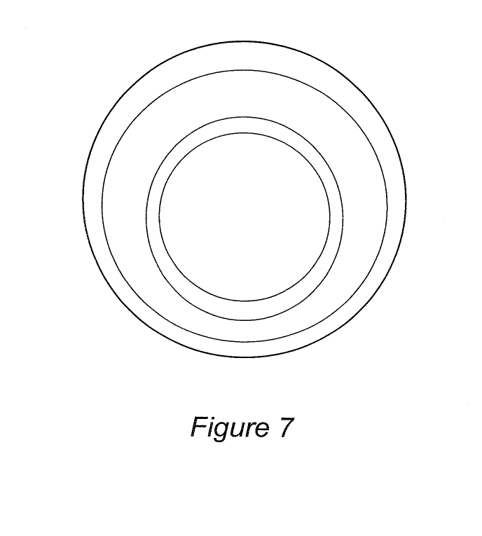

FIG. 7 provides a cross-section at mid length of a coaxial catheter assembly of the present invention in which the venous lumen is nested within the arterial lumen;

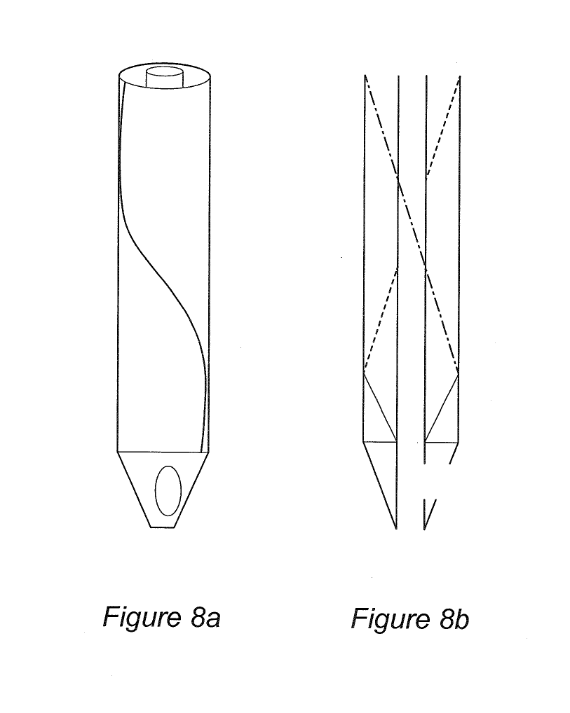

FIGS. 8a and 8b provide views of the distal tip portion of the catheter assembly of FIG. 7 in the insertion position, externally and in cross section respectively;

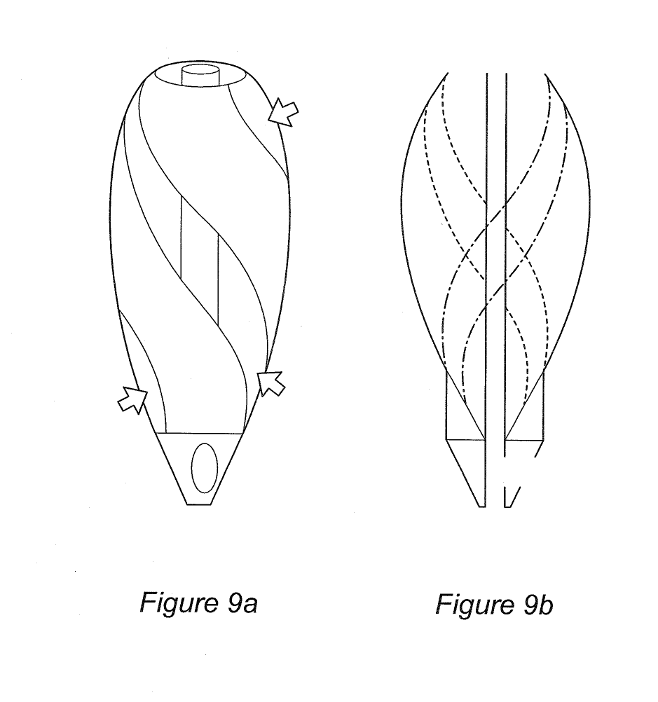

FIGS. 9a and 9b provide views of the distal tip portion of the catheter assembly of FIGS. 7 and 8 in the drawing position, externally and in cross section respectively

FIG. 1 shows a view of a catheter assembly, shown generally at 1, including an arterial lumen 2, a venous lumen 3 and a third lumen 4 housed in a distal tip portion 5. The arterial lumen rotates about and translates along the third lumen to form a first helix. The venous lumen rotates about the third lumen in the opposite direction to the arterial lumen to form a second helix, symmetrical to the first helix about the third lumen. The helix formed by the lumens extends around 25% of the length of the catheter body intended to be positioned within the patient's body in use. There is provided a deployment mechanism 6, through which the arterial lumen 2, the venous lumen 3 and the third lumen 4 extend. The deployment mechanism 6 is a twist mechanism configured to move the third lumen there through and to thus move the arterial and venous lumens from an insertion position (as shown in FIG. 3) to a drawing position (as shown in FIG. 1 and FIG. 2A). Movement from the insertion to the drawing position is effected by twisting the mechanism 6 through 90° to pull the third lumen a controlled distance, generally around 5 mm where the catheter assembly is intended for use on the jugular vein of a human. The proximal ends of the arterial and venous lumens and the third lumen are suitable for attachment to a dialysis machine or to an extracorporeal carbon dioxide removal machine.

According to one embodiment of the catheter assembly of the present invention, there may be provided two similarly dimensioned arterial lumens disposed symmetrically about a venous lumen which extends substantially along the longitudinal axis of the catheter. The venous lumen has an associated cross sectional area around 50% greater than the individual arterial lumens. The two arterial lumens will aspirate, both from the inside of the spiral, and ejection will occur from the centrally situated venous lumen further towards the tip. FIG. 5 shows such a catheter assembly in the drawing position. The arterial apertures face inwards, towards the longitudinal axis of the catheter. The venous apertures are provided closer to the distal tip than the arterial apertures. The venous lumen is pulled back to move the catheter assembly into the drawing position.

In the embodiment illustrated in FIG. 6, the positioning of the venous and arterial lumens have been swapped. The venous apertures are provided closer to the distal tip than the arterial apertures. The arterial lumen is pulled back to move the catheter assembly into the drawing position.

In the embodiment illustrated in FIGS. 7 to 9, there are only two lumens, the venous lumen nested inside the arterial lumen. Helical slits are provided in the external, arterial lumen over the adjustable tip segment. The device is deployed as before, by retracting the venous lumen. In this instance the walls of the arterial lumen bow outwards, exposing the internal aspect of the lumen to the blood stream. FIGS. 9a and 9b illustrate the catheter assembly of FIGS. 7 to 9 in the drawing position. As the venous lumen is retracted, the walls of the arterial lumen splay outwards over the tip segment. There are a minimum of two silts cut into the wall of the arterial lumen. In the closed state these are opposed, and closed. As the venous lumen is retracted, these splay open, exposing the internal aspect of the arterial lumen, and enabling blood flow. Dashed lines in these figures illustrate slit orientations, out of plane of the cross section.

The method of inserting an acute haemodialysis catheter may include the following steps:
a) Insertion of a wire through a needle, into a central vein.
b) Skin incision, and passage of a dilator over the wire into the vein.
c) Removal of the dilator, and passage of the catheter over the wire into the vein.
d) Removal of the wire.

The method for insertion of a chronic catheter may involve the additional formation of a subcutaneous tract via the following steps:
a) Insertion of a wire through a needle, into a central vein.
b) Skin incision beside the wire forming a slit (Slit A)
c) Skin incision at a point around 10 cm away, usually pectoral region (Slit B)
d) Dilation of a subcutaneous tract between slit B, and slit A, through which the catheter is routed.
e) Dilation over the wire into the vein.
f) Either: Passage of the catheter over the wire into the vein, and removal of the wire.
g) Or: Passage of dilator with a tearable sheath. Removal of the wire and dilator, and passage of the catheter into the vein through the sheath. The sheath is then removed.
h) Closure of Slit A by suturing.

The catheter then runs through a subcutaneous tract before penetrating the vein. This is advantageous in reducing infection risk.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A catheter assembly, said catheter assembly comprising a catheter body defining an arterial lumen and a venous lumen, the catheter body including a distal tip portion including or housing a terminal catheter portion, the arterial lumen including at least one aperture at or towards a distal end of the arterial lumen and in fluid communication with the arterial lumen, and the venous lumen including at least one aperture at or towards a distal end of the venous lumen and in fluid communication with the venous lumen,
wherein at least one of the arterial lumen and the venous lumen are radially displaceable in a transverse plane relative to a longitudinal axis of the catheter body between an insertion position and a drawing position; and
wherein a maximum radial displacement in the transverse plane, between the radially displaceable arterial and/or venous lumens and the longitudinal axis of the catheter body is greater in the drawing position than in the insertion position.

2. The catheter assembly as claimed in claim 1, wherein the radially displaceable arterial and/or venous lumens form a helix around the longitudinal axis of the catheter body.

3. The catheter assembly as claimed in claim 1, further comprising:
two lumens radially displaceable in the transverse plane relative to the longitudinal axis of the catheter body; and
one lumen disposed along the longitudinal axis of the catheter body,
wherein the two lumens are substantially symmetrically disposed along the longitudinal axis of the catheter body.

4. The catheter assembly as claimed in claim 3, wherein the two lumens radially displaceable in the transverse plane relative to the longitudinal axis of the catheter body are arterial lumens and the lumen disposed along the longitudinal axis of the catheter body is a venous lumen.

5. The catheter assembly according to claim 1, wherein both of the arterial lumen and the venous lumen are radially displaceable in the transverse plane relative to the longitudinal axis of the catheter body between the insertion position and the drawing position, and the arterial lumen and the venous lumen are symmetrically or substantially symmetrically disposed along the longitudinal axis of the catheter body.

6. The catheter assembly as claimed in claim 5, wherein the arterial and venous lumens are in the form of a double helix around the central longitudinal axis of the catheter body.

7. (canceled)

8. The catheter assembly as claimed in claim 1, wherein one of the venous and arterial lumens is housed within the other of the venous and arterial lumens.

9. (canceled)

10. The catheter assembly as claimed in claim 1, wherein the, or the majority of said aperture(s) of the arterial lumen face within 30° clockwise or anti-clockwise from a cross-sectional plane which intersects the longitudinal axis of the catheter body and a wall of the arterial lumen facing towards the longitudinal axis of the catheter body.

11. (canceled)

12. The catheter assembly as claimed in claim 1, wherein a greatest lateral distance between the arterial lumen and the longitudinal axis of the catheter body towards the distal tip portion is at least two times greater in the drawing position than in the insertion position.

13. The catheter assembly as claimed in claim 1 configured for use in a human, wherein a greatest lateral distance between the arterial lumen and the longitudinal axis of the catheter body towards the distal tip portion is 0.2 to 1.5 cm in the drawing position, and 0.1 cm or less in the insertion position.

14. (canceled)

15. A catheter assembly comprising a catheter body defining an arterial lumen and a venous lumen, the catheter body including a distal tip portion including or housing a terminal catheter portion, the arterial lumen including at least one aperture at or towards a distal end of the arterial lumen and in fluid communication with the arterial lumen, and the venous lumen including at least one aperture at or towards a distal end of the venous lumen and in fluid communication with the venous lumen,
wherein the, or the majority of the aperture(s) at or towards the distal end of the arterial lumen face within 30° (clockwise or anti-clockwise) from a cross-sectional plane which intersects a longitudinal axis of the catheter body and a wall of the arterial lumen facing towards the longitudinal axis of the catheter body.

16. (canceled)

17. The catheter assembly as claimed in claim 15, wherein the arterial lumen and the venous lumen form a double helix around the longitudinal axis of the catheter body in a drawing position.

18. (canceled)

19. The catheter assembly as claimed in claim 17, wherein a helix angle of the arterial and the venous lumen(s) is at least 15° from the longitudinal axis of the catheter body.

20. The catheter assembly as claimed in claim 15, wherein the aperture(s) at or towards the distal end of the venous lumen face 75 to 120° from the cross-sectional plane which intersects the longitudinal axis of the catheter body and a wall of the venous lumen facing towards the longitudinal axis of the catheter body.

21. The catheter assembly as claimed in claim 15, wherein the venous lumen is disposed in a helix around the longitudinal axis of the catheter body, and the arterial lumen is disposed substantially along the longitudinal axis of the catheter body.

22. The catheter assembly as claimed in claim 15, further comprising two venous lumens disposed in a double helix around the longitudinal axis of the catheter body.

23. The catheter assembly as claimed in claim 15, wherein the catheter body defines a third lumen extending from the distal tip portion substantially along the longitudinal axis of the catheter body.

24. (canceled)

25. (canceled)

26. (canceled)

27. (canceled)

28. (canceled)

29. A method for treating blood comprising:
making an incision in a blood vessel,
inserting at least part of the catheter body of the catheter as claimed in claim 1 into the blood vessel, wherein the arterial lumen is provided in the insertion position,
moving the arterial lumen into the drawing position,
drawing blood through the at least one aperture at or towards the distal end of the arterial lumen,
treating the blood,
returning the treated blood to the blood vessel through the at least one aperture at or towards the distal end of the venous lumen.

30. The method of claim 29, wherein the blood is treated through removal of one or more waste products, extra fluid or carbon dioxide.

31. A kit of parts comprising the catheter assembly as claimed in claim 1 and instructions for use.
